(19) United States
(12) Patent Application Publication
(10) Pub. No.: US 2005/0201959 A1
(43) Pub. Date: Sep. 15, 2005

(54) Methods and compositions for altering skin coloration

(75) Inventor: Nathaniel E. David, San Francisco, CA (US)

Correspondence Address: WILSON SONSINI GOODRICH & ROSATI, 650 PAGE MILL ROAD, PALO ALTO, CA, 94304-1050, US

(73) Assignee: VVII NewCo 2003, Inc., Menlo Park, CA (US)

(21) Appl. No.: 10/799,540

(22) Filed: Mar. 11, 2004

(51) Int. Cl. A61K 38/20; A61K 7/135

(52) U.S. Cl. 424/62; 424/85.2

(57) ABSTRACT

Novel compositions and methods and pharmaceutical compositions for altering skin coloration. The methods include administering a cytokine to a dermal region desired to be altered. The cytokine is formulated for local administration. The cytokine is preferably administered in conjunction with a therapeutic procedure. The therapeutic procedure is preferably selected from the group consisting of excision, dermabrasion, laser therapy, cryosurgery, grafting, camouflaging, scarification, and salabrasion.

### BACKGROUND OF THE INVENTION

Skin color is a conspicuous way in which humans vary. Today, many people use tattoos to alter skin coloration for aesthetic and cosmetic reasons. For example, some individuals tattoo permanent makeup. Others use tattooing to simulate natural pigmentation. Tattooing can also be used as part of an initiation ceremony to a social group.

Whatever the reason is, tattooing has become a common procedure. It is approximated that over 10 million Americans have at least one tattoo, and that close to 4,000 tattoo studios currently operate in the United States. Yet, estimates suggest that almost 50 percent of all those who get tattoos later decide to remove them.

Tattoo removal can be painful, expensive and often results in scarring or discoloration of the skin. The most commonly used color alteration procedures these days are excision, dermabrasion, laser therapy, cryosurgery, grafting, camouflaging, scarification, and salabrasion. However, no matter which procedure is used, the average tattoo requires 8-12 treatments before it is substantially removed.

Thus, it is desirable to identify novel methods and compositions to reduce the number of treatments for tattoo removal, alleviate the pain associated with tattoo removal, and enhance the results.

### SUMMARY OF THE INVENTION

The present invention involves methods and compositions for altering skin coloration, and, in particular, tattoo removal. In preferred embodiments, the methods herein provide administering to a dermal region an effective amount of a cytokine (e.g., a tumor necrosis factor, interferon, or interleukin). The cytokine administered is preferably not a GM-CSF. The cytokine administered is preferably a tumor necrosis factor, an interferon, or an interleukin. More preferably, the cytokine administered is TNF-α, IFN-α, and/or IL-1.

One or more cytokines is preferably administered locally. Local administration is preferably made by topical, subcutaneous, or transdermal administration. The cytokines can be administered as a single dose, multiple doses, in combination with other agents, and/or in combination with other treatments.

In some embodiments, the dermal region being treated with a cytokine is also treated with a color alteration treatment. Examples of color alteration treatments include, but are not limited to, excision, dermabrasion, laser therapy, cryosurgery, grafting, camouflaging, scarification, and salabrasion. In preferred embodiments, the color alteration treatment is a laser therapy. In some embodiments, the cytokine is administered prior to the color alteration treatment. In some embodiments, the cytokine is administered after the color alteration treatment. In some embodiments, the cytokine is administered during a color alteration treatment.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates signaling pathways of the immune system.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

Changes in skin coloration can be caused by numerous biological and non-biologic factors. Non-biologic factors that can cause alterations in skin colorations include tattoos. The word tattoo comes from the Tahitian “tatu” which means “to mark something”. It is arguably claimed that tattooing has existed since 12,000 years BC. Three examples of tattoos include: decorative tattoos, traumatic tattoos and gunpowder tattoos. Decorative tattoos are made by repeatedly puncturing of the skin with a needle saturated with colored ink. Traumatic tattoos can occur, for example, if the skin is grazed along the surface of a road and tiny pieces of grit and carbon powder enter the skin. Gunpowder explosions can cause tattooing if the gunpowder penetrates the skin.

Today, decorative tattooing is very common. It is approximated that over 10 million Americans have at least one tattoo, and that close to 4,000 tattoo studios currently operate in the United States. Many people use tattoos to alter skin coloration for aesthetic and cosmetic reasons. For example, some individuals tattoo permanent makeup (e.g., on eyelids, lips, eyebrows, etc.) to save time or because they have physical difficulty applying regular, temporary makeup. Tattooing can also be an addition or substitution to reconstructive surgery, particularly of the face or breast, to simulate natural pigmentation. In some instances, people who have lost their eyebrows due to alopecia (a form of hair loss) may choose to have “eyebrows” tattooed on, while others with vitiligo (a lack of pigmentation in areas of the skin) may try tattooing to help camouflage the condition. Furthermore, tattooing can be part of an initiation right (e.g., to a fraternity or a gang).

Tattooing involves rapidly and repeatedly injecting ink into the dermal layer of the skin with a small needle to develop a permanent coloration. A small tattoo takes about 45 minutes and a larger one may take many hours or repeated visits. The inks used by most tattoo artists are not really inks but rather pigments that are suspended in a carrier solution. The pigments are usually not vegetable dyes. Instead, today's pigments are primarily metal salts. However, some pigments are plastics and there are some vegetable dyes that are used as well. The pigment provides the color of the tattoo. The purpose of the carrier is to disinfect the pigment suspension, keep it evenly mixed, and provide for ease of application.

The pigment, grit, carbon or ink used for tattooing is considered a food additive by the Food and Drug Administration (FDA) and causes minimal adverse reactions. Under a microscope, tattoos appear as tiny granules of color pigment. Tattoo granules are initially dispersed in the upper dermis and vertical foci at sites of injection. Approximately 7-14 days after injection, the granules concentrate at a more focal location. Tattoo granules are composed of loosely packed particles, ranging from approximately 2-400 nm in diameter. The most common particle size is about 40 nm. Less common particle sizes are about 2-4 nm is size and about 350-400 nm is size.

Tattoo granules are endocytosed by fibroblasts as well as macrophages in the dermis and subcutis. Normally, foreign bodies are attacked and removed from the body by the natural defense mechanism of macrophage activity. However, tattoo particles are sufficiently large to inhibit activity by macrophages and tattoo pigment, grit, carbon or ink remains in the skin. This results in an appearance of macrophage “freezing.” See Fujita H, Arch. Histol. Cytol. (1988) Jul; 51(3):285-94. Thus, a tattoo is relatively permanent.

The oldest pigments came from using ground up minerals and carbon black. Today's pigments include the original mineral pigments, modem industrial organic pigments, a few vegetable-based pigments, and some plastic-based pigments. Allergic reactions, scarring, phototoxic reactions (i.e., reaction from exposure to light, especially sunlight), and other adverse effects are possible with many pigments. The plastic-based pigments are very intensely colored, but there are many reported adverse reactions to them. Recently, there has been development of pigments that glow in the dark or in response to black (ultraviolet) light. While some of these pigments may be safe, others are radioactive or otherwise toxic. Below is a table listing some commonly used pigments in tattoo inks. This list is not exhaustive. Just about anything can be used as a pigment. Also, many inks mix one or more pigment:

TABLE 1
Commonly used compositions in tattoo inks

| Final Color | Material Used |
|---|---|
| Black | Iron Oxide (Fe₃O₄); Iron Oxide (FeO); Carbon; Logwood |
| Brown | Ochre |
| Red | Cinnabar (HgS); Cadmium Red (CdSe); Iron Oxide (Fe2O3); Napthol-AS |
| | pigment |
| Orange | disazodiarylide and/or disazopyrazolone; cadmium seleno-sulfide |
| Flesh | Ochres (iron oxides mixed with clay) |
| Yellow | Cadmium Yellow (CdS, CdZnS); Ochres; Curcuma Yellow; Chrome |
| | Yellow (PbCrO4, often mixed with PbS); disazodiarylide |
| Green | Chromium Oxide (Cr₂O₃), called Casalis Green or Anadomis Green; |
| | Malachite [Cu₂(CO₃)(OH)₂]; Ferrocyanides and Ferricyanides; Lead |
| | chromate; Monoazo pigment; Cu/Al phthalocyanine; Cu phthalocyanine |
| Blue | Azure Blue; Cobalt Blue; Cu-phthalocyanine |
| Violet | Manganese Violet (manganese ammonium pyrophosphate); Various |
| | aluminum salts; Quinacridone; and Dioxazine/carbazole |
| White | Lead White (Lead Carbonate); and Titanium dioxide (TiO₂) |
| | Barium Sulfate (BaSO₄); and Zinc Oxide |

Until recently, government has not attempted to regulate the use of tattoo inks and the pigments used in them. However, with the growing popularity of tattooing and permanent makeup, the U.S. federal drug agency has begun looking at safety issues concerning tattoo removal, adverse reactions to tattoo colors, and infections that result from tattooing.

Beyond the pain often associated with getting a tattoo, there are numerous risks involving both tattooing and removal of a tattoo. These risks include infection, allergic reactions, granulomas, keloid formation, MRI complications and removal problems. Infection is common and can be avoided by using clean needles and sterile ink. Allergic reactions to tattoo pigments are rare. However, when they do occur they may be particularly troublesome, especially because the pigments may be hard to remove. Thus, it may be desirable to remove a tattoo due to an allergic reaction to the pigment or ink. Granulomas are nodules that may form around material such as tattoo ink that the body perceives as foreign. If and when a granuloma is formed, it may be desirable to quickly remove to the tattoo. Keloid formation are scars that grow beyond normal boundaries. Keloids may form from an injury or trauma to the skin. Tattooing (and tattoo removal) can cause keloid formations especially in individuals who are susceptible to such formations. Additional complication associated with tattoos include reports that people with tattoos or permanent makeup who experienced swelling or burning in the tattooed areas when they undergo magnetic resonance imaging (MRI). This seems to occur only rarely and apparently without lasting effects. However, there are also reports that tattoo pigments can interfere with the quality of the image. This seems to occur mainly when a person with permanent eyeliner undergoes MRI of the eyes.

The most common reason people with tattoos seek medical care is that they want the tattoo removed. Conservative estimates suggest that almost 50 percent of all people who get tattoos later decide to remove them. Despite advances in laser technology, tattoo removal is a painful process that usually involves multiple treatments and a considerable expense. Complete removal without scarring may be impossible. Currently, there are several methods for tattoo removal. The most popular of these methods include: excision, dermabrasion, laser therapy, cryosurgery, grafting, camouflaging, scarification, and salabrasion.

Excision involves an injection of a local anesthetic to numb the area after which the tattoo is removed surgically. The edges are then brought together and sutured. With this procedure, there is minimal bleeding which is easily controlled with electrocautery. In some cases involving large tattoos, a skin graft taken from another part of the body may be necessary. Excision sometimes involves the use of tissue expanders (balloons inserted under the skin, so that when the tattoo is cut away, there is less scarring). Larger tattoos may require repeated surgery for complete removal.

Dermabrasion, which is usually used for smaller tattoos, involves spraying the tattoo with a solution that freezes the area. The tattoo is then “sanded” with a rotary abrasive instrument causing the skin to peel. Because some bleeding is likely to occur, a dressing is immediately applied to the area.

Laser therapy is a popular technique for tattoo removal. Commonly used lasers include the Versapuls C with helper H laser, Q-switched Nd:YAG (532 nm, 1064 nm), Q-switched alexandrite (855 mm), and the Q-switched ruby (694 nm). Recent developments in laser therapy involve the development of picosecond lasers. The present invention contemplates all other lasers. Q-switched ruby and alexandrite lasers are useful for removing black, blue, and green pigments. The Q-switched 532 nm Nd:YAG laser can be used to remove red pigments and the 1064 Nd:YAG laser is used to remove black and blue pigments. Thus, often time more than one wavelength or laser is used to remove a multi-colored tattoo. After the tattoo area is numbed, pulses of light from a laser are directed onto the tattoo. The laser breaks up the tattoo pigment, and subsequently, the body's scavenger cells remove the treated pigmented areas. Generally, several visits are necessary over a span or weeks or months, and the treatments can be expensive. Some individuals experience hypopigmentation—a lightening of the natural skin coloring—in the affected area. Laser treatments also can cause some tattoo pigments to change to a less desirable shade.

Cryosurgery is the freezing of tissue prior to its removal or excision. Grafting involves removing a skin graft taken from another part of the body to cover a tattooed region. Scarification involves removing the tattoo with an acid solution and creating a scar in its place. Camouflaging a tattoo entails the injection of new pigments either to form a new pattern or cover a tattoo with skin-toned pigments. However, it is noted that injected pigments may not to appear natural because they lack the skin's natural translucence.

Salabrasion is a procedure similar to demabrasion in which the tattooed area is first numbed with a local anesthesia. Subsequently, a solution of ordinary tap water dipped in table salt is applied to the area, and an abrading apparatus such as the one used with dermabrasion, or an even simpler device such as a wooden block wrapped in gauze, is used to vigorously abrade the area. When the area becomes deep red in color, a dressing is applied.

Regardless of which technique is used, tattoo removal generally results in textual changes, scarring, and discoloration. In rare cases, localized and generalized allergic reaction can occur. The effectiveness of tattoo removal depends on various factors, including but not limited to, the size of the tattoo, the location of the tattoo, the individual's healing process, how the tattoo was applied, and the length of time that the tattoo has been on the skin. A tattoo performed by a more experienced tattoo artist, for example, may be easier to remove since the pigment is evenly injected in the same level of the skin. A tattoo that has been on the skin for a considerable length of time may be more difficult to remove than a new one.

Preliminary results from a recent animal study suggest that topical imiquimod 5% cream used in the acute phase after tattooing may have utility as a nonsurgical method for pigment removal. See Dermatol. Surg., 28(1) (2002); see also Derm. Times, 22(4) (2001), both of which are incorporated herein by reference for all purposes. This study involved five albino guinea pigs that were tattooed with black, red, green and yellow dye. A punch biopsy was taken with 6 hours after tattooing. Then one animal served as control and the others were allocated to one of four treatments: petrolatum, tretinoin 0.025 percent, imiquimod 5 percent cream, and tretinoin alternated with imiquimod. Each agent was applied every 6 hours for seven days, and the responses were evaluated clinically, and with repeat biopsies at seven and 28 days after tattoo placement. Macroscopically and histologically, imiquimod alone appeared to be the most effective regimen for fading the tattoo. However, the biopsy evaluation also revealed the presence of epidermal and dermal necrosis with separation, severe inflammation and fibrosis, and disruption of the skin appendages at the imiquimod-treated site.

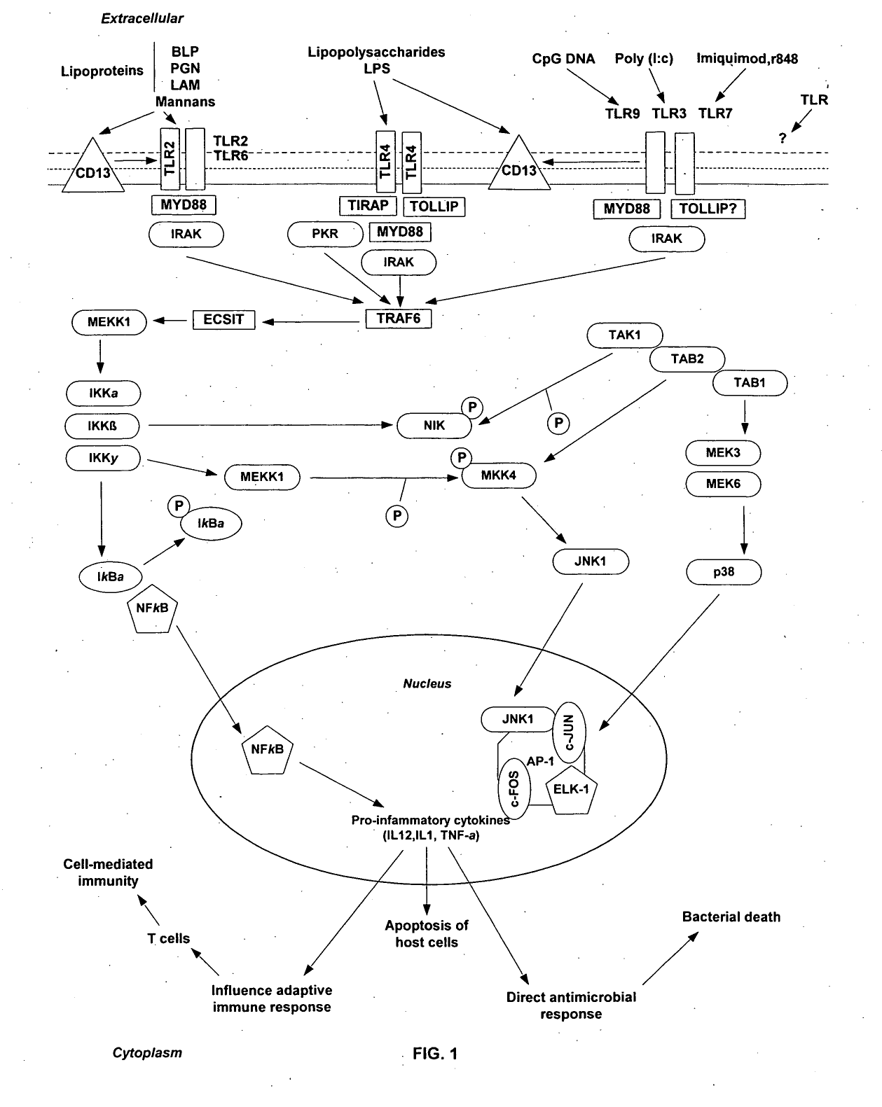

Imiquimod is a small molecule, which is a toll-like receptor (TLR) agonist that is capable of indirectly activating multiple arms of the innate immune response. FIG. 1 illustrates the indirect activation of the immune system by imiquimod. In particular, imiquimod binds TLR-7 on the cell surface and generates a signal via the TRAF6 pathway. This signaling pathway leads to the nucleus of the cell via the p38, JNK1, or NF-kB MAP kinase pathways. Activation of the above signaling pathways induces the production of pro-inflammatory cytokines, including but not limited to TNF-α, Interferon-α, and IL-1.

Thus, the present invention contemplates the local and direct administration of cytokines as a means for altering skin coloration. The term “cytokine” as used herein refers to any substance produced by cells that has a specific effect on cell-cell interaction, communication and/or behavior of other cells. More preferably, a cytokine is any substance released by cells that has a specific effect on cell-cell interaction, communication and/or behavior of other cells. In some embodiments, a cytokine is a small protein or a biological factor. Preferably, a cytokine is in the range of 1-40 kD, more preferably 2-30 kD, more preferably 3-20 kD, or more preferably 4-25 kD. In preferred embodiments, a cytokine is selected from the group consisting of interleukins, lymphokines, tumor necrosis factors, interferons, chemokines, and growth factors.

Interleukins are secretory proteins produced by lymphocytes, monocytes and other cells types. Interleukins are often released by cells in response to antigenic and non-antigenic stimuli. Examples of interleukins include, but are not limited to, IL-1 through IL-15. In preferred embodiments, a cytokine of the present invention is IL-1 or IL-2, or any homologs, derivatives, variants, or mimetics thereof. More preferably, a cytokine of the present invention is IL-1, or any homologs, derivatives, variants, or mimetics thereof.

Lymphokines are soluble factors that are secreted by activated lymphocytes and that affect other lymphocytes and other cell types. Representative examples of lymphokines include, but are not limited to, IL-1 through IL-15, GM-CSF, G-CSF, M-CSF, alpha.-, beta.-, or gamma-interferon, tumor necrosis factors, and their respective receptors. In preferred embodiments, a lymphokine is selected from the group consisting of a CSF receptor, alpha-interferon, interleukins-2 or any homologs, derivatives, variants, or mimetics thereof. More preferably, a lymphokine is interferon-α, or any homologs, derivatives, variants, or mimetics thereof.

Tumor necrosis factors are cytokines produced mainly by macrophages and T lymphocytes that help regulate the immune response and hematopoiesis (blood cell formation). Examples of tumor necrosis factors include: TNF-α (also called cachectin) and TNF-β (also called lymphotoxin). TNF-α is produced by macrophages, while TNF-β is produced by activated CD4+ T cells. In preferred embodiments, a cytokine of the present invention is TNF-α or any homologs, derivatives, variants, or mimetics thereof.

Interferons are glycoproteins derived from human cells that normally play a role in fighting viral infections by preventing virus multiplication in cells. There are multiple types of interferons (e.g., Type I and Type II). Examples of interferon Type I cytokines include, but are not limited to, interferon-α and interferon-β. Examples of interferon Type II cytokines include, but are not limited to, interferon-γ. Preferably, a cytokine of the present invention is interferon-α or any homolog, derivative, variant, or mimetic thereof.

Chemokines are cytokines that are chemotactic for leucocytes. Chemokines can be subdivided into two general groups on the basis of the arrangement of a pair of conserved cysteines: the C×C group includes platelet Factor 4, platelet basic protein, IL-8, melanoma growth stimulatory protein, and macrophage inflammatory protein 2. The C C group, on the other hand, include, but are not limited to, TECK, TARC, RANTES, MIP-1, MCP-1, MCP-3, MCP-4, MDS, MIP-1, MIP-3, MIP-4, Eotaxin-1, Eotaxin-2, and Exodus-1.

Growth factors are substances produced by a leucocyte that acts upon another cell. Examples are interleukins, interferon-alpha, lymphotoxin, tumor necrosis factors, erythropoietin (epoietin-α), and colony-stimulating factors (CSFs). Colony-stimulating factors stimulate production of white blood cells (WBCs). Examples of CSFs include, but are not limited to, granulocyte-CSF (C-CSF) (e.g., filgrastin), and granulocyte macrophage-CSF (GM-CSF) (e.g., sargramostim). Examples of commercial embodiments of CSFs include, but are not limited to, Leukine™, Neupogen™ and Neulasta™. Each of the above CSFs varies slightly in its effect on the body and in the indications for which they are marketed for usage. In preferred embodiments, the cytokine of the present invention is a growth factor but not a CSF. In other embodiments, the cytokine of the present invention is a CSF selected from the group consisting of Leukine™, Neupogen™ and Neulasta™.

In addition to cytokines, the present invention also contemplates the use of substances that stimulate or enhance cytokine production. Examples of substances that stimulate or enhance cytokine production include, but are not limited to, flagellum (stimulating CSFs), Echinacea, endothelins, vitamin A, vitamin B5, anti-oxidants, etc.

In particular the present invention contemplates the local administration of one or more substances (cytokine or substance that induces or enhances cytokine production) to alter skin coloration. Such substances are preferably administered to a dermal region desirable of being of a different color. In some embodiments, the dermal region includes a tattooed region. The tattoo can be, for example, a decorative tattoo, a traumatic tattoo, or a gunpowder tattoo, and it may be desirous to either change the coloration of the tattoo or to remove or reduce the coloration from the tattoo.

In one example, a dermal skin region desirable of being of a different color is a decorative tattoo having one of more pigments. The pigments can be any one of the pigments disclosed herein or any other pigments, whether or not approved for tattoo use.

The methods for altering skin coloration disclosed herein include administering, preferably locally, to the dermal skin region desirable of being of a different skin color one or more of the compounds disclosed herein. In preferred embodiments, a compound administered is a cytokine. More preferably, a compound administered is an interleukin, an interferon, or a tumor necrosis factor. More preferably, a compound administered is IL-1, INF-α, or TNF-α.

When the methods are used to remove or reduce a tattoo, administration of any of the compound herein can occur, for example, immediately after injection of a pigment into the skin (e.g., a mistake by a tattoo artist) or after a prolonged period (e.g., due to an individual's desire to have the tattoo removed). This approach, because it selectively activates only a single art of the immune system, may have fewer side effects and thus better safety to efficacy performance than direct imiquimod application (which activates multiple arms of the innate immune response).

The compounds of the present invention can be part of a kit, which can include one or more cytokines, individually packaged. A kit for skin color alteration would typically comprise at least one compound such as a cytokine or a substance that enhances cytokine production. Preferably, the kit will also contain instructions for proper use and disposal of the contents after use. The instruction can include, for example, a description as to which compound should be used to achieve a particular result (e.g., color alteration) and how to administer the compound.

The compounds of the present invention can be administered by any suitable route, preferably in the form of a pharmaceutical composition adapted to such a route, and in a dose effective for the treatment intended. The active compounds and composition can, for example, be administered orally, intravascularly (IV), intraperitoneally, subcutaneously, intramuscularly (IM) or topically including by way of a patch. In preferred embodiments, the active compounds of the present invention are administered topically to the tattooed region.

The compounds of the present invention can also be administered by injection (IV, IM, subcutaneous or jet) as a composition wherein, for example, saline, dextrose, or water can be used as a suitable carrier. The pH value of the composition can be adjusted, if necessary, with suitable acid, base, or buffer. Suitable bulking, dispersing, wetting or suspending agents, including mannitol and PEG 400, can also be included in the composition. A suitable parenteral composition can also include a compound formulated as a sterile solid substance, including lyophilized powder, in injection vials. Aqueous solution can be added to dissolve the compound prior to injection.

A pharmaceutical composition can contain any compound disclosed herein at any thereapeutically effective amount. Preferably a pharmaceutical composition contains about 0.1 to 1000 mg of a compound (e.g., a cytokine or a substance that enhances cytokine production), more preferably at about 7.0 to 350 mg of a compound, more preferably about 15 to 250 mg of a compound, or more preferably about 20 to 150 mg of a compound. The compounds herein can be administered once per treatment cycle or multiple times per treatment cycle. For example, single or multiple doses can be made prior to, during, or after each color alteration treatment.

In some embodiments, a topical preparation of the compounds herein are applied to the tattooed area 1-10 times a day, more preferably 1-5 times a day, or more preferably 1-3 times a day, and are preferably applied as a topical gel, spray, ointment or cream containing the active ingredients in a total amount of, for example, 0.075 to 30% w/w, preferably 0.2 to 20% w/w and most preferably 0.4 to 15% w/w.

The compounds can be applied prior to, during, or post a color alteration treatment. More preferably, the compounds are applied prior to a color alteration treatment. A color alteration treatment is any procedure (whether chemical, physical, biological, etc.) known by a person of ordinary skill in the art that is used to reduce, alter, or eliminate skin coloration, whether such skin coloration is naturally occurring (e.g., freckles) or non-naturally occurring (e.g., a tattoo). Examples of color alteration treatments include, but are not limited to, excision, dermabrasion, laser therapy, cryosurgery, grafting, camouflaging, scarification, and salabrasion. In preferred embodiments, the color alteration treatment is laser therapy. The compounds herein can be administered prior to, during, and/or post a color (e.g., tattoo) alteration treatment.

In one embodiment, coloration resulting from a tattoo is wholly or partially removed by administering one or more of the compounds disclosed herein to the tattooed dermal region. Such compounds are preferably administered locally, (e.g., topically or transdermally). The compounds are preferably administered prior to or during a color alteration treatment, wherein the color alteration treatment is preferably laser therapy.

When formulated as an ointment, the active ingredients (cytokines) can be employed, for example, with either paraffinic or a water miscible ointment base. Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof.

The topical formulation can desirably include a compound that enhances absorption or penetration of the active ingredient through the skin or other affected areas. Examples of such dermal penetration enhancers include dimethylsulfoxide and related analogs.

The compounds of this invention can also be administered by a transdermal device. Preferably, topical administration is accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent can also function as the membrane. The transdermal patch can include the compound in a suitable solvent system with an adhesive system, such as an acrylic emulsion, and a polyester patch.

The effective amount of compounds administered and doses will vary depending on the patient's natural skin color, coloration desirous of being removed, added or altered, size of target region desirable of having a different coloration, the location of the target region, and the color alteration treatment used in conjunction with the cytokines.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for altering coloration of a dermal region comprising administering to said region an effective amount of an interleukin.

2. The method of claim 1 wherein said dermal region comprises a tattoo.

3. The method of claim 2 wherein said tattoo is selected from the group consisting of a decorative tattoo, a traumatic tattoo, a gunpowder tattoo.

4. The method of claim 1 wherein said dermal region comprises a decorative tattoo.

5. The method of claim 1 wherein said dermal region comprises a traumatic tattoo.

6. The method of claim 1 wherein said dermal region comprises a gunpowder tattoo.

7. The method of claim 1 wherein said altering comprises reducing the effective amount of said coloration.

8. The method of claim 1 wherein said interleukin is selected from the group consisting of IL-1, IL-2, IL-3, IL-4, IL-5, IL-6, IL-7, IL-8, IL-9, IL-10, IL-11, IL-12, IL-13, IL-14, and IL-15.

9. The method of claim 1 wherein said interleukin is administered 1-10 times a day.

10. The method of claim 1 wherein said interleukin is administered topically or subcutaneously.

11. The method of claim 1 wherein said interleukin is administered transdermally.

12. The method of claim 1 further comprising a color alteration treatment.

13. The method of claim 12 wherein said color alteration treatment is selected from the group consisting of excision, dermabrasion, laser therapy, cryosurgery, grafting, camouflaging, scarification, and salabrasion.

14. The method of claim 13 wherein said color alteration treatment is laser therapy.

15. The method of claim 14 wherein said laser therapy is preformed with a Q-switched Nd:YAG laser, a Q-switched alexandrite laser, or a Q-switched ruby laser.

16. The method of claim 15 wherein said interleukin is administered prior to said color alteration treatment.

17. The method of claim 16 wherein said interleukin is administered prior to or post said color alteration treatment.

18. The method of claim 17 wherein said interleukin is administered prior to said laser therapy.

19. The method of claim 8 wherein said interleukin is IL-1.

20. A method for altering coloration of a dermal region comprising administering to said region an effective amount of a tumor necrosis factor.

21. The method of claim 20 wherein said dermal region comprises a tattoo.

22. The method of claim 21 wherein said tattoo is selected from the group consisting of a decorative tattoo, a traumatic tattoo, a gunpowder tattoo.

23. The method of claim 20 wherein said dermal region comprises a decorative tattoo.

24. The method of claim 20 wherein said dermal region comprises a traumatic tattoo.

25. The method of claim 20 wherein said dermal region comprises a gunpowder tattoo.

26. The method of claim 20 wherein said altering comprises reducing the effective amount of said coloration.

27. The method of claim 20 wherein said tumor necrosis factor is TNF-alpha or TNF-beta.

28. The method of claim 20 wherein said tumor necrosis factor is administered 1-10 times a day.

29. The method of claim 20 wherein said tumor necrosis factor is administered topically or subcutaneously.

30. The method of claim 20 wherein said tumor necrosis factor is administered transdermally.

31. The method of claim 20 further comprising a color alteration treatment.

32. The method of claim 31 wherein said color alteration treatment is selected from the group consisting of excision, dermabrasion, laser therapy, cryosurgery, grafting, camouflaging, scarification, and salabrasion.

33. The method of claim 32 wherein said color alteration treatment is laser therapy.

34. The method of claim 33 wherein said laser therapy is preformed with a Q-switched Nd:YAG laser, a Q-switched alexandrite laser, or a Q-switched ruby laser.

35. The method of claim 34 wherein said tumor necrosis factor is administered prior to said color alteration treatment.

36. The method of claim 35 wherein said tumor necrosis factor is administered prior to or post said color alteration treatment.

37. The method of claim 36 wherein said tumor necrosis factor is administered prior to said laser therapy.

38. The method of claim 27 wherein said tumor necrosis factor is TNF-alpha.

39. A method for altering coloration of a dermal region comprising administering to said region an effective amount of an interferon.

40. The method of claim 39 wherein said dermal region comprises a tattoo.

41. The method of claim 39 wherein said tattoo is selected from the group consisting of a decorative tattoo, a traumatic tattoo, a gunpowder tattoo.

42. The method of claim 40 wherein said dermal region comprises a decorative tattoo.

43. The method of claim 40 wherein said dermal region comprises a traumatic tattoo.

44. The method of claim 40 wherein said dermal region comprises a gunpowder tattoo.

45. The method of claim 39 wherein said altering comprises reducing the effective amount of said coloration.

46. The method of claim 39 wherein said interferon is selected from the group consisting of interferon-alpha, interferon-beta, and interferon-gamma.

47. The method of claim 39 wherein said interferon is administered 1-10 times a day.

48. The method of claim 39 wherein said interferon is administered topically or subcutaneously.

49. The method of claim 39 wherein said interferon is administered transdermally.

50. The method of claim 39 further comprising a color alteration treatment.

51. The method of claim 39 wherein said color alteration treatment is selected from the group consisting of excision, dermabrasion, laser therapy, cryosurgery, grafting, camouflaging, scarification, and salabrasion.

52. The method of claim 51 wherein said color alteration treatment is laser therapy.

53. The method of claim 52 wherein said laser therapy is preformed with a Q-switched Nd:YAG laser, a Q-switched alexandrite laser, or a Q-switched ruby laser.

54. The method of claim 53 wherein said interferon is administered prior to said color alteration treatment.

55. The method of claim 54 wherein said interferon is administered prior to or post said color alteration treatment.

56. The method of claim 55 wherein said interferon is administered prior to said laser therapy.

57. The method of claim 39 wherein said interferon is interferon-alpha.

58. A method for altering coloration of a dermal region comprising administering to said region an effective amount of a cytokine excluding a macrophage colony-stimulating factor.

59. The method of claim 58 wherein said dermal region comprises a tattoo.

60. The method of claim 59 wherein said tattoo is selected from the group consisting of a decorative tattoo, a traumatic tattoo, a gunpowder tattoo.

61. The method of claim 59 wherein said altering comprises reducing the effective amount of said coloration.

62. The method of claim 59 wherein said cytokine is selected from the group consisting of interferon-alpha, IL-1, and TNF-alpha.

63. The method of claim 59 wherein said cytokine is administered 1-10 times a day.

64. The method of claim 59 wherein said cytokine is administered topically or subcutaneously.

65. The method of claim 59 wherein said cytokine is administered transdermally.

66. The method of claim 59 further comprising a color alteration treatment.

67. The method of claim 66 wherein said color alteration treatment is selected from the group consisting of excision, dermabrasion, laser therapy, cryosurgery, grafting, camouflaging, scarification, and salabrasion.

68. The method of claim 67 wherein said color alteration treatment is laser therapy.

69. The method of claim 68 wherein said laser therapy is preformed with a Q-switched Nd:YAG laser, a Q-switched alexandrite laser, or a Q-switched ruby laser.

70. The method of claim 66 wherein said cytokine is administered prior to said color alteration treatment.

71. The method of claim 66 wherein said cytokine is administered prior to or post said color alteration treatment.

72. The method of claim 68 wherein said cytokine is administered prior to said laser therapy.
